(19) United States
(12) Patent Application Publication
(10) Pub. No.: US 2005/0215800 A1
(43) Pub. Date: Sep. 29, 2005

(54) Method for producing gamma-butyrolactone

(76) Inventors: Rolf Pinkos, Bad Durkheim (DE); Rolf-Hatmuth Fischer, Heidelberg (DE); Markus Rosch, Oppenheim (DE); Nils Bottke, Mannheim (DE); Stephan Schlitter, Ludwigshafen (DE); Michael Hesse, Worms (DE); Alexander Weck, Freinsheim (DE); Gunther Windecker, Ludwigshafen (DE); Gunnar Heydrich, Limburgerhof (DE)

Correspondence Address: Keil & Weinkauf, 1350 Connecticut Avenue N W, Washington, DC, 20036, US

(21) Appl. No.: 10/513,040

(22) PCT Filed: Apr. 25, 2003
(86) PCT No.: PCT/EP03/04287

§ 371 Date: Nov. 1, 2004

(30) Foreign Application Priority Data
Apr. 30, 2002 (DE) 102 19 224.3

(51) Int. Cl. C07D 307/26

(52) U.S. Cl. 549/325

(57) ABSTRACT

The present invention relates to a process for preparing unsubstituted or C₁-C₄-alkyl-substituted gamma-butyrolactone by catalytic hydrogenation of maleic acid and/or its derivatives in the presence of chromium-free catalyst comprising from 10 to 80% by weight of copper oxide and from 10 to 90% by weight of at least one catalyst support selected from the group consisting of silicon dioxide, titanium dioxide, hafnium dioxide, magnesium silicate, activated carbon, silicon carbide, zirconium dioxide and alpha-aluminum oxide.

The present invention relates to a process for preparing unsubstituted or substituted C₁-C₄-alkyl-substituted gamma-butyrolactone by catalytic hydrogenation of maleic acid and its derivatives in the gas phase. For the purposes of the present patent application, derivatives of maleic acid are esters and anhydrides, which, like the acid, may bear one or more C₁-C₄-alkyl substituents. A catalyst comprising from 10 to 80% by weight of copper oxide and from 10 to 90% by weight of at least one catalyst support selected from the group consisting of silicon dioxide, titanium dioxide, hafnium dioxide, magnesium silicate, activated carbon, silicon carbide, zirconium dioxide and alpha-aluminum oxide is used.

The preparation of gamma-butyrolactone (GBL) by gas-phase-hydrogenation of maleic anhydride (MA) is a reaction which has been known for many years. Numerous catalyst systems are described in the literature for carrying out this catalytic reaction.

A catalyst made up exclusively of copper oxide and aluminum oxide for the gas-phase hydrogenation of MA to produce GBL is disclosed in WO 97/24346. This catalyst comprises from 50 to 95%, preferably from 80 to 90%, of copper oxide, from 3 to 30% by weight, preferably from 5 to 15% by weight, of aluminum oxide and optionally a binder. In the hydrogenation of MA using such a catalyst over a period of 1600 hours, the GBL yield was 92-93% by weight. The high copper contents of the catalysts adversely affect the economics due to high material costs.

EP 404 808 discloses coated catalysts having a catalytically active composition of the formula Cu₁Zn_{b}Al_{c}M_{d}Oₓ, where M is at least one element selected from the group consisting of the elements of groups IIA to IVA, group VIII, Ag, Au, groups IIIB, VIIB and the rare earths, for catalyzing the hydrogenation of MA to form mixtures of GBL and THF. The catalytically active composition is applied to an essentially inert, at least partly porous support having an external surface. Compared to the unsupported catalyst, which gives THF as main product, this coated catalyst gives GBL as preferentially produced product. In Example 3, for example, a GBL yield of 91.3% is achieved using a catalyst of the composition CuAl_{1.2}ZnCr_{0.004}Oₓ at a pressure of 2 bar, 280° C., a hydrogen/MA ratio of 100 and a GHSV (Gas hourly space velocity) of 11420 h. A disadvantage is that, here too, chromium is present in all the catalysts used in the examples. Furthermore, large amounts of succinic anhydride are formed.

Chromium-free catalyst systems for preparing GBL by hydrogenation of MA are known, for example, from WO 99/35139. The optionally supported catalysts disclosed comprise, as catalytically active composition, from 50 to 90% of CuO and from 10 to 50% of ZnO. In Example 2, the reaction is carried out in the presence of a catalyst comprising 64% by weight of CuO and 23.5% by weight of ZnO. In this example, GBL yields of from 94.9 to 96.6% are achieved at from 245 to 270° C. and 5 bar.

All the catalysts described in the abovementioned publications have the disadvantage that they give undesirable by-product. The catalysts frequently also contain chromium. In addition, the catalyst operating lives which can be achieved are unsatisfactory.

It is an object of the present invention to provide a process by means of which unsubstituted or C₁-C₄-alkyl-substituted GBL can be prepared by hydrogenation of maleic acid and its derivatives, with both MA and its derivatives being able to bear one or more C₁-C₄-alkyl substituents. This process should be able to be carried out continuously using chromium-free catalysts and give very large amounts of the desired product GBL so as to achieve the best possible economics. Furthermore, the catalyst should be able to be employed when MA which has not been subjected to costly prepurification is used and nevertheless have a long operating life, i.e. not require frequent regeneration.

We have found that this object is achieved by a process for preparing gamma-butyrolactone (GBL) by catalytic hydrogenation of maleic acid and/or its derivatives in the presence of a chromium-free catalyst comprising from 10 to 80% by weight of copper oxide (CuO) and from 10 to 90% by weight of at least one catalyst support selected from the group consisting of silicon dioxide, titanium dioxide, hafnium dioxide, magnesium silicate, activated carbon, silicon carbide, alpha-aluminum oxide and zirconium dioxide.

The process of the present invention can be operated in an inexpensive manner and gives high GBL yields and selectivities. It can be carried out continuously, which is preferred according to the present invention.

An important aspect of the present invention is the choice of the catalyst which comprises copper oxide as main catalytically active constituent. This copper oxide is applied to a support which has to have a small number of acid centers. Large numbers of acid centers in the support material, for example in the gamma-aluminum oxide, favor the formation of tetrahydrofuran as undesirable by-product. The catalyst used according to the present invention is therefore free of acidic gamma-aluminum oxide. Suitable support materials having a small number of acid centers are silicon oxide, titanium dioxide, hafnium dioxide, magnesium silicate, activated carbon and silicon carbide or mixtures thereof, of which silicon dioxide, zirconium dioxide, titanium dioxide, activated carbon and alpha-aluminum oxide are particularly preferred.

The amount of copper oxide is <80% by weight. Preferred catalyst compositions comprise <70% by weight of copper oxide and >10% by weight of support, and particularly preferred catalysts comprise from 10 to 65% by weight of copper oxide and from 35 to 90% by weight of support. Low copper oxide contents are preferred because of the cost advantage which is achieved thereby.

The catalysts used according to the present invention, which are free of chromium, can optionally further comprise from 0 to 10% by weight, preferably from 0.1 to 5% by weight, of one or more further metals or one or more metal compounds, preferably an oxide, of elements selected from the group consisting of silver, molybdenum, tungsten, rhodium, manganese, rhenium, ruthenium, palladium and platinum, of which manganese, rhenium, palladium and silver are preferred.

Furthermore, the catalysts used according to the present invention may optionally further comprise up to 50% by weight, preferably up to 20% by weight, of a metal or a metal compound, preferably an oxide, of zinc, of an element of group 1 or 2 (IA and IIA according to the old IUPAC nomenclature) of the Periodic Table of the Elements and/or of the rare earth metals including the lanthanides.

Preferred elements which may be present in the catalysts used according to the present invention are zinc, lithium, sodium, potassium, rubidium, cesium, magnesium, calcium, strontium, barium, scandium, yttrium, lanthanum and the rare earth elements having atomic numbers from 58 to 71. Particular preference is given to sodium, potassium, calcium, magnesium, lanthanum and zinc.

In addition, the catalysts used can contain an auxiliary in an amount of from 0 to 10% by weight. For the purposes of the present invention, auxiliaries are organic and inorganic substances which contribute to improved processing during catalyst production and/or to an increase in the mechanical strength of the shaped catalyst bodies. Such auxiliaries are known to those skilled in the art; examples include graphite, stearic acid, silica gel and/or copper powder.

The catalysts can be produced by methods known to those skilled in the art. Preference is given to processes in which the copper oxide is obtained in finely divided form and is intimately mixed with the other constituents.

These starting materials can be processed to form the shaped bodies by known methods, for example extrusion, tabletting or by agglomeration processes, if appropriate with addition of auxiliaries.

Catalysts able to be used according to the present invention can also be produced by, for example, application of the active components to a support, for example by impregnation or vapor deposition. Furthermore, catalysts which are able to be used according to the present invention can be obtained by shaping a heterogeneous mixture of active component or precursor compound thereof with a support component or precursor compound thereof.

In the hydrogenation according to the present invention, in which not only MA but also other C₄-dicarboxylic acids or derivatives thereof defined below can be used as starting material, the catalyst is used in reduced, activated form. Activation is carried out using reducing gases, preferably hydrogen or hydrogen/inert gas mixtures, either before or after installation in the reactor in which the process of the present invention is carried out. If the catalyst is installed in the reactor in oxidic form, activation can be carried out either before the plant is started up to carry out the hydrogenation of the present invention or during start-up, i.e. in situ. The separate activation before start-up of the plant is generally carried out by means of reducing gases, preferably hydrogen or hydrogen/inert gas mixtures, at elevated temperatures, preferably from 100 and 300° C. In in-situ activation, activation is carried out during running-up of the plant by contact with hydrogen at elevated temperature.

The catalysts are used as shaped bodies. Examples include rods, ribbed rods, other extruded shapes, pellets, rings, spheres and crushed material.

The BET surface area of the copper catalysts in the oxidic state is from 10 to 400 m²/g, preferably from 15 to 200 m²/g, in particular from 20 to 150 m²/g. The copper surface area (N₂0 decomposition) of the reduced catalyst in the installed state is >0.2 m²/g, preferably >1 m²/g, in particular >2 m²/g.

In one variant of the invention, catalysts having a defined porosity are used. These catalysts have, as shaped bodies, a pore volume of ≧0.01 ml/g for pore diameters of >50 nm, preferably ≧20.025 ml/g for pore diameters of >100 nm and in particular ≧0.05 ml/g for pore diameters of >200 nm. Furthermore, the ratio of macropores having a diameter of >50 nm to the total pore volume for pores having a diameter of >4 nm is >10%, preferably >20%, in particular >30%. The porosities mentioned were determined by mercury intrusion in accordance with DIN 66133. The data in the pore diameter range from 4 nm to 300 nm were evaluated.

The catalysts used according to the present invention generally have a satisfactory operating life. Should the activity and/or selectivity of the catalyst nevertheless drop during the period of operation, this can be restored by means of measures known to those skilled in the art. These include, for example, reductive treatment of the catalyst in a stream of hydrogen at elevated temperature. If appropriate, the reductive treatment can be preceded by an oxidative treatment. Here, a gas mixture comprising molecular oxygen, for example air, is passed through the catalyst bed at elevated temperature. Furthermore, it is possible to wash the catalyst with a suitable solvent, for example ethanol, THF or GBL, and subsequently to dry it in a gas stream.

For the purposes of the present patent application, the term maleic acid and its derivatives as starting materials refers to maleic acid and succinic acid, which may bear one or more C₁-C₄-alkyl substituents, and also the esters and anhydrides of these unsubstituted or alkyl-substituted acids. An example of such an acid is citraconic acid. Preference is given to using the anhydride of a given acid. The use of MA as starting material is particularly preferred.

The process of the present invention can be operated inexpensively, achieves high GBL yields of up to 95% and more and gives pure GBL after a simple work-up.

The process can be carried out batchwise or continuously, preferably continuously.

In addition, adherence to particular reaction parameters is necessary to achieve the GBL yields obtained according to the present invention.

An important parameter is adherence to a suitable reaction temperature. This can be achieved by a sufficiently high inlet temperature of the starting materials. This is in the range from >180° C. to 300° C., in particular from >220 to 300° C., preferably 240 to 290° C. To obtain an acceptable or high GBL selectivity and yield, the reaction has to be carried out so that an appropriately high reaction temperature prevails in the catalyst bed over which the actual reaction takes place. This hot spot temperature is established after entry-of the starting materials into the reactor and is in the range from 240 to 320° C., preferably from 240 to 300° C.

The molar ratio of hydrogen/starting material is likewise a parameter which has an important influence on the product distribution and also on the economics of the process of the present invention. From an economic point of view, a low hydrogen/starting material ratio is desirable. The lower limit is about 3, but higher molar ratios of hydrogen/starting material of from 20 to 600 can generally be employed. The use of the above-described catalysts and adherence to the above-described temperatures allows the use of favorable, low hydrogen/starting material ratios, preferably in the range from 20 to 200, more preferably from 40 to 150. The most favorable range is from 50 to 100. The heat evolved in the reaction is in this case removed by internal heat removal. Alternatively, it is possible to employ hydrogen:MA ratios of from 400:1 to 600:1. In this case, the heat is carried from the reactor by the circulating gas. An inexpensive shaft reactor can be used in this case.

To set the molar ratio of hydrogen/starting material used according to the present invention, part of the hydrogen, advantageously the major part of the hydrogen, is circulated. This is generally done using recycle gas compressors known to those skilled in the art. The hydrogen consumed chemically by the hydrogenation is replaced. In a preferred embodiment, part of the circulating gas is discharged to remove inert compounds, for

example n-butane. The circulated hydrogen can also be used, if appropriate after preheating, for vaporizing the starting material stream.

The volume flow of the reaction gases, generally expressed as GHSV (Gas Hourly Space Velocity), is also an important parameter in the process of the present invention. The GHSV values in the process of the present invention are in the range from 100 to 20,000 standard m³/m³h, preferably from 1000 to 3000 standard m³/m³h, in particular from 1100 to. 2500 standard m³/m³h.

The pressure at which the hydrogenation according to the present invention is carried out is in the range from 1 to 30 bar, preferably from 1 to 15 bar, in particular from 1 to 10 bar.

All products which do not condense or condense only incompletely on cooling the gas stream leaving the hydrogenation reactor are circulated together with the circulating hydrogen. These are, in particular, water and by-products such as THF, methane and butane. The temperature to which the product stream is cooled is from 0 to 60° C., preferably from 20 to 45° C. The THF content of the circulating gas is from 0.1 to 5% by volume, in particular from 0.5 to 3% by volume.

The hydrogenation of MA to give GBL proceeds via the intermediate succinic anhydride (SA). The process of the present invention makes it possible to hydrogenate MA and derivatives thereof to give reaction mixtures which contain little THF and, accordingly, 95 mol% of GBL (based on MA) or more. The GBL yield can be influenced, in particular, by the choice of hydrogenation catalyst, the space velocity over the catalyst, the hydrogenation temperature, the reaction pressure and the molar ratio of starting material/hydrogen. The amount of THF increases with increasing number of acid centers in the catalyst, with decreasing space velocity over the catalyst, with rising temperature and with increasing reaction pressure.

Possible types of reactor are all apparatuses which are suitable for heterogeneously catalyzed reactions using a gaseous starting material stream and product stream. Preference is given to tube reactors, shaft reactors or reactors having internal heat removal, for example shell-and-tube reactors, and the use of a fluidized bed is also possible. Preference is given to using shell-and-tube reactors.

The gas stream leaving the reactor is cooled to from 10 to 100° C. The reaction products are condensed out in this way and are passed to a separator. The uncondensed-gas stream is taken off from the separator and conveyed to the recycle gas compressor. A small amount of circulating gas is discharged. The reaction products which have condensed out are continuously taken from the system and passed to work-up. The by-products present in the liquid phase which has been condensed out are mainly water and n-butanol together with small amounts of propanol, methanol, ethanol, n-butyraldehyde, butyl methyl ether and further oxygen-containing compounds whose concentration is below 200 ppm.

In the process of the present invention, starting materials to be hydrogenated of differing purity can be used in the hydrogenation reaction. Of course, it is possible to use a starting material of high purity, in particular MA, in the hydrogenation reaction. However, the catalyst used according to the present invention and also the other reaction conditions selected according to the present invention make it possible to use starting materials, in particular MA, which are contaminated with the usual compounds obtained in the oxidation of benzene, butenes or n-butane and possibly further components. Thus, the hydrogenation process of the present invention can, in a further embodiment, include an upstream stage comprising the preparation of the starting material to be hydrogenated by partial oxidation of a suitable hydrocarbon and separation of the starting material to be hydrogenated from the resulting product stream.

In particular, this starting material to be hydrogenated is MA. Preference is given to using MA which comes from the partial oxidation of hydrocarbons. Suitable hydrocarbon streams are benzene, C₄-olefins (e.g. n-butenes, C₄ raffinate streams) or n-butane. Particular preference is given to using n-butane, since it is an inexpensive, economical starting material. Processes for the partial oxidation of n-butane are described, for example, in Ullmann's Encyclopedia of Industrial Chemistry, 6^{th} Edition, Electronic Release, Maleic and Fumaric Acids—Maleic Anhydride.

The crude reaction product obtained in this way is then taken up in a suitable organic solvent or solvent mixture which has a boiling point at atmospheric pressure which is at least 30° C. higher than that of MA.

This solvent (absorption medium) is brought to a temperature in the range from 20 to 160° C., preferably from 30 to 80° C. The MA-containing gas stream from-the partial oxidation can be brought into contact with the solvent in a variety of ways: (i) passing the gas stream into the solvent (e.g. via gas inlet nozzles or sparging rings), (ii) spraying the solvent into the gas stream and (iii) bringing the upflowing gas stream into countercurrent contact with the downflowing solvent in a tray column or packed column. In all three variants, the apparatuses known to those skilled in the art for gas absorption can be used. In choosing the solvent to be used, it should be ensured that it does not react with the starting material, for example the MA which is preferably used. Suitable solvents are: tricresyl phosphate, dibutyl maleate and dibutyl fumarate, dimethyl maleate and dimethyl fumarate, high molecular weight waxes, aromatic hydrocarbons having a molecular weight of from 150 to 400 and a boiling point above 140° C., for example dibenzylbenzene; dialkyl phthalates having C₁-C₈-alkyl groups, for example dimethyl phthalate, diethyl phthalate, dibutyl phthalate, di-n-propyl phthalate and diisopropyl phthalate; di-C₁-C₄-alkyl esters of other aromatic and aliphatic dicarboxylic acids, for example dimethyl naphthalene-2,3-dicarboxylate, dimethyl cyclohexane-1,4-dicarboxylate, methyl esters of long-chain fatty acids having, for example, from 14 to 30 carbon atoms, high-boiling ethers, for example dimethyl ethers of polyethylene glycol, for example tetraethylene glycol dimethyl ether. In place of diesters, it is also possible to use the corresponding monoesters. Preferred examples are monoalkyl maleates and monoalkyl fumarates, in particular the methyl and butyl esters.

The use of phthalates is preferred.

The solution which results after the treatment with the absorption medium generally has an MA content of from about 5 to 400 gram per liter.

The offgas stream which remains after treatment with the absorption medium comprises mainly the by-products of the preceding partial oxidation, e.g. water, carbon monoxide, carbon dioxide, unreacted butanes, acetic acid and acrylic acid. The offgas stream is virtually free of MA.

The dissolved MA is subsequently stripped from the absorption medium. This is carried out using hydrogen at a pressure which is equal to or at most 10% above the pressure in the subsequent hydrogenation or alternatively under reduced pressure with subsequent condensation of remaining MA. In the stripping column, a temperature profile resulting from the boiling points of MA at the top and the virtually MA-free absorption medium at the bottom of the column at the respective column pressure and the dilution with carrier gas employed (in the first case with hydrogen) is observed.

To prevent losses of solvent, rectification internals can also be located above the feed point for the crude MA stream. The virtually MA-free absorption medium taken off at the bottom is returned to the absorption zone. In the case of direct stripping with hydrogen, a virtually saturated gas stream of MA in hydrogen is taken off at the top of the column. In the other case, the condensed MA is pumped into a vaporizer and there vaporized into the circulating gas stream.

The MA/hydrogen stream further comprises by-products which are formed in the partial oxidation of n-butane, butenes or benzene by means of oxygen-containing gases, and also absorption medium which has not been separated off. These components are, in particular, acetic acid and acrylic acid as by-products, water, maleic acid and the dialkyl phthalates which are preferably used as absorption medium. The MA contains from 0.01 to 1% by weight, preferably from 0.1 to 0.8% by weight, of acetic acid and from 0.01 to 1% by weight, preferably from 0.1 to 0.8% by weight, of acrylic acid, based on MA. In the hydrogenation stage, acetic acid and acrylic acid are at least partly hydrogenated to ethanol and propanol, respectively. The maleic acid content is from 0.01 to 1% by weight, in particular from 0.05 to 0.3% by weight, based on MA.

If dialkyl phthalates are used as absorption medium, the amount present in the MA is critically dependent on correct operation of the stripping column, in particular the enrichment section. Phthalate contents of up to 1.0% by weight, in particular up to 0.5% by weight, should not be exceeded in appropriate operation, since the consumption of absorption medium otherwise becomes too high.

The hydrogen/maleic anhydride stream obtained in this way is then fed to the hydrogenation zone and hydrogenated as described above. The catalyst activity and catalyst operating life are in this case virtually unchanged from those obtained when using MA which has been substantially prepurified, for example by distillation.

The hydrogenation product which has been condensed out is then worked up by distillation to give GBL which meets the required specification.

### EXAMPLES

### Example 1

a) Production of the catalyst

Catalyst A:

Catalyst A was produced by impregnating 5 mm extrudates comprising a mixture of 92% by weight of SiO₂ and 8% of CaO with a solution of copper carbonate in concentrated aqueous ammonia. Impregnation was carried out by spraying the copper carbonate solution onto the extrudates for 15 minutes. The impregnated extrudates were dried at 120° C. for 3 hours and then calcined at 350° C. for 2 hours. The finished catalyst A comprises 13% by weight of CuO, 7% of CaO and 80% of SiO₂ (calculated on the basis of 100% oxides).

Catalyst B:

Catalyst B was produced by impregnating SiO₂ spheres having a diameter of 3-5 mm with a solution of copper carbonate in concentrated aqueous ammonia. Impregnation was carried out in solution covering the spheres for 15 minutes. The impregnated spheres were dried at 120° C. for 5 hours and then calcined at 250° C. for 2 hours. These impregnation and calcination steps were repeated a number of times. The finished catalyst B comprises 25.6% by weight of CuO and 74.4% of SiO₂ (calculated on the basis of 100% oxides).

Catalyst B

(25% by weight of CuO, 75% by weight of SiO₂) was produced analogously.

b) Catalyst Activation

Before commencement of the reaction, the catalyst was subjected to treatment with hydrogen in a hydrogenation apparatus. For this purpose, the reactor was brought to 240 or 265° C. and the catalyst was activated for the time indicated in Table 1 with the mixture of hydrogen and nitrogen indicated in each case at atmospheric pressure.

TABLE 1

| Time | Temperature | Hydrogen | Nitrogen |
|---|---|---|---|
| (minutes) | [° C.] | (standard l/h) | (standard l/h) |
| 15 | 240 | 500 | 10 |
| 10 | 240 | 500 | 20 |
| 20 | 240 | 400 | 40 |
| 120 | 240 | 200 | 65 |
| 720 | 265 | 0 | 65 |

c) Hydrogenation Apparatus

The pressure apparatus used for the hydrogenation comprised a vaporizer, a reactor, a condenser with quench feed, a hydrogen inlet, an offgas line and a recycle gas blower. The pressure in the apparatus was kept constant.

The molten MA was pumped from above onto the preheated (245° C.) vaporizer and vaporized. A mixture of fresh hydrogen and circulated gas was likewise fed onto the vaporizer from above. In this way, hydrogen and MA entered the heated reactor from below. The reactor was packed with a mixture of glass rings and catalyst. After the hydrogenation, the resulting GBL together with water, other reaction products and hydrogen left the reactor and was condensed in the cooler by quenching. Part of the circulating gas was discharged before the remainder, mixed with fresh hydrogen, reentered the vaporizer.

The condensed liquid reaction-product, the offgas and the circulating gas were analyzed quantitatively by gas chromatography.

### Examples 2 and 3

Hydrogenation of maleic anhydride prepared from n-butane.

The reactor of the hydrogenation apparatus described in Example 1c was charged with 220 ml of the catalyst A prepared as described in Example 1 a and 126 ml of Raschig rings. Activation was carried out as described in Example 1 b.

The starting material used was maleic anhydride prepared from n-butane. No deactivation of the catalyst, i.e. no decrease in the maleic anhydride conversion and/or the gamma-butyrolactone yield, was observed during the reaction. Table 2 summarizes the reaction parameters in the hydrogenation and the results.

### Example 4

Catalyst B having the composition 25% by weight of CuO, 74.4% by weight of SiO₂ was used in place of catalyst A. After activation of the catalyst, it too was used for the hydrogenation of maleic anhydride prepared from n-butane without deactivation being observed during the reaction. Table 2 summarizes the reaction parameters in the hydrogenation and the results.

TABLE 2

| | | | | Through- | | | | | | |
|---|---|---|---|---|---|---|---|---|---|---|
| | | Temperature | Pressure | put | H₂/MA | Conversion | GBL | BDO | THF | SA |
| Ex. | Catalyst | [° C.] | [bar] | [kg_{MA}/kg_{cat}h] | [mol/mol] | [%] | [%] | [%] | [%] | [%] |
| 2 | A | 280 | 5.4 | 0.15 | 115 | 100 | 94 | 0.3 | 1 | 0 |
| 3 | A | 267 | 15 | 0.1 | 126 | 100 | 84 | 0.7 | 6.2 | 0.2 |
| 4 | B | 260 | 5.3 | 0.1 | 93 | 100 | 93 | 0.4 | 1.3 | 0 |

BDO = 1,4-Butanediol
SA = Succinic anhydride

1. A process for preparing unsubstituted or C₁-C₄-alkyl-substituted gamma-butyrolactone by catalytic hydrogenation of maleic acid and/or its derivatives in the presence of chromium-free catalyst comprising from 10 to 80% by weight of copper oxide, up to 50% by weight of at least one metal or metal compound of elements of groups 1 and 2 of the Periodic Table of the Elements and/or the rare earth metals including the lanthanides and from 10 to 90% by weight of at least one catalyst support selected from the group consisting of silicon dioxide, titanium dioxide, hafnium dioxide, magnesium silicate, activated carbon, silicon carbide, zirconium dioxide and alpha-aluminum oxide.

2. A process as claimed in claim 1, wherein the catalyst further comprises up to 10% by weight of one or more further metals or compounds thereof selected from the group consisting of silver, molybdenum, tungsten, manganese, rhodium, ruthenium, rhenium, palladium and platinum.

3. A process as claimed in claim 1 carried out at pressures of from 1 to 30 bar.

4. A process as claimed in claim 1 wherein the molar ratio of hydrogen/starting material is in the range from 20 to 600.

5. A process as claimed in claim 1 wherein the GHSV is in the range from 100 to 20,000 standard m³/m³h.

6. A process as claimed in claim 1 wherein the inlet temperature is in the range from >180 to 300° C.

7. A process as claimed in claim 1 wherein the catalyst is activated by reduction, preferably by treatment with hydrogen or a hydrogen/inert gas mixture, before or after it is installed in the reactor and before it is used in the hydrogenation reaction.

8. A process as claimed in claim 1 wherein the catalyst contains an auxiliary, preferably graphite, stearic acid, silica gel and/or copper powder, in an amount of <10% by weight.

9. A process as claimed in claim 1 wherein the shaped catalyst body has a pore volume of ≧0.01 ml/g for pore diameters of >50 nm.

10. A process as claimed in claim 1 wherein the ratio of macropores having a diameter of >50 nm to the total pore volume for pores having a diameter of >4 nm in the shaped catalyst body is >10%.

11. A process as claimed in claim 1 wherein maleic anhydride which has been prepared by oxidation of benzene, C₄-olefins or n-butane is used, where the crude maleic anhydride obtained by oxidation has been extracted from the crude product mixture by means of a solvent and has subsequently been stripped from this solvent by means of hydrogen.
